(19) United States
(12) Patent Application Publication
(10) Pub. No.: US 2010/0311846 A1
(43) Pub. Date: Dec. 9, 2010

(54) METHODS FOR CONTROLLING WATER AMOUNT IN A POLYMER COMPOSITION OR SUBSTRATE

(76) Inventors: Matthew Bendiner, Pinehurst, NC (US); Matthew Dyer, Pinehurst, NC (US); Carolyn M. Merkel, North Haledon, NJ (US)

Correspondence Address: MYERS BIGEL SIBLEY & SAJOVEC, PO BOX 37428, RALEIGH, NC, 27627, US

(21) Appl. No.: 12/793,150

(22) Filed: Jun. 3, 2010

Related U.S. Application Data
(60) Provisional application No. 61/184,949, filed on Jun. 8, 2009.

(51) Int. Cl. C08K 5/09; D21H 17/14; A23D 9/00; A24B 15/30; A61K 47/12; C09K 3/00; B05D 1/02

(52) U.S. Cl. 514/784; 524/300; 162/179; 426/601; 131/290; 252/194; 427/421.1

(57) ABSTRACT

A method of controlling the amount of water in a polymer composition or substrate is provided. The method includes the step of adding to the polymer composition or substrate a low molecular weight unsaturated fatty acid which optionally includes a stabilizer composition for preventing oxidation of the low molecular weight unsaturated fatty acid.

### CROSS-RELATED APPLICATION

This application claims priority from U.S. Provisional Patent Application No. 61/184,949; filed Jun. 8, 2009, the disclosure of which is incorporated herein by reference in its entirety.

### FIELD OF THE INVENTION

This invention relates to methods of controlling the amount or level of water in polymer compositions or substrates.

### BACKGROUND OF THE INVENTION

The ability to control moisture amounts or levels in a product is critical for a wide range of practical operations. Products as diverse as edible snack cakes and concrete mortar rely on management of short- and long-term moisture transport phenomena for processing control as well as extension of shelf life (Slade & Levine, 1995). Of particular interest is the ability to control moisture amounts in the pulp and paper industries as well as food industries. This interest is related to the fact that the energy costs of water removal in finished products can be a significant driver of overall product cost (Department of Energy).

Methods to improve dewatering of materials are known. For example, in paper processing, water may be removed by gravity/drainage at the forming stage, mechanical means at the pressing stage, and energy (heat) introduction at the drying stage. Many techniques for dewatering have focused on improving the removal of water in the drainage stage where the cost of water removal is lowest. For example, a method is described in U.S. Pat. No. 4,795,531 to Sofia, et al. in which a low molecular weight cationic organic polymer (molecular weight between 2000 and 200,000) is combined with an acrylamide copolymer of molecular weight greater than 500,000 to facilitate removal of water at the forming stage.

U.S. Pat. No. 5,942,086 to Owen describes a method of sequential addition of a cationic polymeric flocculent (molecular weight greater than 150,000) to a paper slurry, followed by the addition of an anionic polyhydroxy high molecular weight polymer. A gel is formed which allows for improved finished paper performance concomitant with an improvement in dewatering at the forming stage.

U.S. Pat. No. 7,189,776 to Carr and Sigman describes the addition of anionic organic polymeric particles along with colloidal anionic silica-based particles with surface areas of 300-100 m²/g to improve drainage in cellulosic suspensions. In common with previously disclosed methods, dewatering is improved by the addition of high molecular polymeric materials, and dewatering improvement is found at the forming stage.

Food materials are also typically dried using a multi-stage process. For example, corn wet gluten is milled, then first dewatered using drainage, often assisted by the use of a centrifuge. Then the material is mechanically dewatered typically with a vacuum, and then final dewatering is accomplished by addition of energy such as in a drying oven. U.S. Pat. No. 5,840,850 to Parlady provides for the use of anionic surfactants, preferably sulfates and sulfonates, to improve dewatering of gluten in the vacuum removal stage. U.S. Patent Publication No, 2009/0005539 by Scheimann and Kowalski proposes the use of anionic polymers to improve the dewatering of corn gluten at the mechanical dewatering stages.

Of particular interest are materials and methods for dewatering at the final phase of the drying process, where the water can be especially difficult to remove. It is desirable that such materials and methods are inexpensive, widely available and safe for use across broad categories of product types. Additionally, it is desirable for such materials or methods to provide a functional benefit in the finished material.

### SUMMARY OF THE INVENTION

The present invention provides a method of controlling the amount of water in a polymer composition or substrate. The method comprises the step of adding to the polymer composition or substrate a low molecular weight unsaturated fatty acid which optionally includes a stabilizer composition for preventing oxidation of the low molecular weight unsaturated fatty acid.

The present invention also provides a method of reducing the amount of water in a polymer composition. The method comprises the steps of adding to the polymer composition a low molecular weight unsaturated fatty acid and optionally a stabilizer composition for preventing oxidation of the low molecular weight unsaturated fatty acid, and subjecting the polymer composition to conditions sufficient to remove water from the polymer composition.

The resulting polymer composition or substrate has substantially reduced amounts of water. Additionally such polymer compositions or substrates are resistant to water adsorption during storage.

### BRIEF DESCRIPTION OF THE DRAWINGS

FIG. 1 measures Time of Heating versus % Remaining Moisture Lost in Example 2.

FIG. 2 measures Time of Heating versus % Remaining Moisture Lost in Example 3.

FIG. 3 measures Time of Heating versus % Remaining Moisture Lost in Example 4.

FIG. 4 measures Time of Heating versus % Remaining Moisture Lost in Example 5.

FIG. 5 measures Time of Heating versus % Remaining Moisture Lost in Example 6.

FIG. 6 measures the effect of potassium sorbate on Zeta potential in Example 6.

FIG. 7 measures Time of Heating versus % Remaining Moisture Lost in Example 7.

### DETAILED DESCRIPTION OF THE INVENTION

The present invention now will be described more fully hereinafter. The invention may, however, be embodied in many different forms and should not be construed to be limited to the embodiments set forth herein; rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items.

Unless otherwise defined, all technical and scientific terms used herein have the same meaning as commonly understood by one of ordinary skill in the art to which the invention belongs.

All publications, U.S. patent applications, U.S. patents and other references cited herein are incorporated by reference in their entireties.

A low molecular weight unsaturated fatty acid may be added to a polymer composition or substrate to control the amount of water in the polymer composition or substrate. The low molecular weight unsaturated fatty acid may be comprised of sorbic acid and/or its salts. Optionally the low molecular weight unsaturated fatty acid may be stabilized. The stabilization may be accomplished by the use of a stabilizer such as manganous ion to help prevent oxidation of the sorbic acid and its salts, preventing the development of aldehydes, ketones and the like. (See, for example, U.S. Pat. No. 5,354,902).

According to the embodiments of the invention, the low molecular weight unsaturated fatty acid is added in an amount which is sufficient to provide control of the amount of water in a polymer composition or a water-containing substrate. It is understood that the phrase “provide control of the amount of water” can have a wide variety of meanings including providing control so that all or substantially all water is removed, providing control so that the removal of water is done more rapidly, with less energy, more efficiently, and the like, and providing control so that less water is re-adsorbed once water is removed. Also it is to be understood that water exists in materials in many forms. It may be entirely free, in that almost no energy input is required to remove the water from the system but water can be readily added back. The water may also be not bound to the system, but require some mechanical assistance such as centrifugation or pressing to remove the water, particularly if rapid removal is desired. The water may be bound to the matrix in some way that energy, typically thermal energy in the form of heat, is required to remove the water from the system. Lastly, water may be a component of the system, such as a water of hydration in a crystal.

The amount of low molecular weight unsaturated fatty acid in solution may be about 0.001% to about 50% by weight of the total weight of solution. In some embodiments, the salt of the low molecular weight unsaturated fatty acid is a potassium salt sorbate and the corresponding amount in solution may be about 0.001% to about 67% by weight of the total weight of solution. The potassium salt sorbate is desirable in some embodiments because of its solubility in aqueous solution and its non-hazardous nature, which is generally recognized and used in various products intended for human and animal use. Other salts may include sodium, calcium, and magnesium. It is understood by those skilled in the art that the weight percent of each salt may be adjusted to assure delivery of the effective level of the low molecular weight unsaturated fatty acid in solution.

The amount of the stabilizer depends upon the conditions required for controlling the amount of water, e.g., dewatering. For example, when the pH of the product is high (i.e., pH>7) and the added concentration of the low molecular weight unsaturated fatty acid is also high, sufficient low molecular weight unsaturated fatty acid may remain to allow for improved dewatering without addition of chemical stabilizer. However, when the pH is lower or the concentration of the low molecular weight unsaturated fatty acid is lower, then a stabilizer composition in the way of, for example, manganous ion, is preferred. It will be recognized by those of ordinary skill in the art that the constituent concentrations may be adjusted depending upon the exact conditions of controlling the amount or level of water.

The stabilized composition may be added in a liquid form. In some embodiments the stabilized composition is an aqueous solution. In other embodiments, the stabilized composition may be a solution of a polar solvent such as methanol or ethanol, or may be combinations of partly or fully miscible solvents such that at least one solvent is a polar solvent such as water.

According to the embodiments of the invention, the stabilizer composition (e.g., manganous ion) may be added to the solution in any manner recognized by one of ordinary skill in the art. For example, the manganous ion may be added to the solution by using a soluble manganous salt such as manganese sulfate. For sorbic acid, the level of manganous ion should be in the weight ratio between 8000:1 to 5;1 sorbate ion to manganous ion, it may be about 4000:1 and often is about 1000:1. Such an amount will allow for complete dissolution of the manganous salt, while providing sufficient ion to stabilize the sorbic acid against decomposition. In general, the stabilizer ensures the efficiency of the low molecular weight unsaturated fatty acid solution over a long period of time and conditions including heat.

According to the embodiments of the invention, the composition of the invention can be applied to moisture-containing products having between 1 and 99% solids. In some embodiments the level of solids may be between 65 and 99%. In another embodiment, the composition of the invention can be applied to products in pH ranges from about 3 to 11.

The composition of the invention may include a surfactant. For example, propylene glycol may be added to the solution to achieve even dispersion when the solution is sprayed upon a moist solid. Other surfactants including but are not limited to sodium dodecyl sulfate, benzalkonium chloride cetyl alcohol or yucca may be used in appropriate systems. The surfactant may be added at a level of between about 0.5 to 10%, and may often be between about 1% to 2%. According to the embodiments of the invention, the surfactant may be used for surface applications when the low molecular weight unsaturated fatty acid solution is sprayed, for example, in a fine layer.

Suitable substrates include low-moisture wood products and pulp, including cardboard and paper products, animal feed including alfalfa hay, distiller's grain, gypsum board, textiles, pharmaceuticals, concrete, cereals, pet food, snack foods, produce, dairy products, unprocessed and processed agricultural foods including corn gluten, packaged human foods, tobacco, rubber, specialty polymers, charcoal, coal and minerals, bakery products and the like. The method of application to the substrate may be a spray pump, although other techniques such as brush application, roller application, dipping, and the like, will be apparent to those skilled in the art.

Suitable polymer compositions include any thermoplastic or thermosetting polymer compositions including polysaccharides such as cellulose, lignin or chitin, polypeptides such as silk, keratin, hair or nylon, or condensation polymers such as polyesters, polyamides and polyacetals, and the like.

The following Examples illustrate various embodiments of the present invention but are not meant to be limiting in any way.

### Example 1

A comparison experiment was conducted between a buffered propionic acid preservative (available as Baler Plus®, AgResearch, Joilet, Ill.) and stabilized potassium sorbate solution (3% potassium sorbate, 0.006% manganous ion) on intermediate-moisture (19% at cutting) alfalfa hay. Both treatments were added to sample products at the rate of 12 pounds per ton of hay. The treated hay was then placed in a hay shed and stacked according to usual practice. After 28 days composite samples were taken with a hay sampling probe and submitted to a commercial testing laboratory for analysis. The results of the test are shown in Table 1.

TABLE 1

| Attribute | Propionic Acid Treated | Sorbic Acid Treated |
|---|---|---|
| Moisture (%) | 15.47 | 14.26 |
| Relative Feed Value* | 131 | 132 |
| Yeasts* | 1.0 × 10⁵ | ND^{#} |
| Molds* | 2.7 × 10⁷ | 9.0 × 10⁶ |

*Dry weight basis,
^{#}None Detected

The comparison experiment showed that the sample product treated with potassium sorbate was lower in moisture, after storage, than a similarly conventionally treated sample. The potassium sorbate treated sample also maintained a functional benefit in terms of yeast and mold reduction compared with the conventionally treated sample.

### Example 2

Four test samples comprising 42% potassium sorbate and 0.084% MnSO₄, 10% potassium sorbate and 0.02% MnSO₄, 5% potassium sorbate and 0.01% MnSO₄, and 3% potassium sorbate and 0.006% MnSO₄ were generated to form Example 2. Chargemaster Cationic Starch R430 from Grain Processing Corporation was added to each test sample. A control composition comprising water and Chargemaster Cationic Starch R430 from Grain Processing Corporation was also prepared. Chargemaster Cationic Starch R430 was added at the same level to the test samples as well as the control, with the level of cationic starch sufficient to treat each test at 6 pounds of starch per ton of pulp.

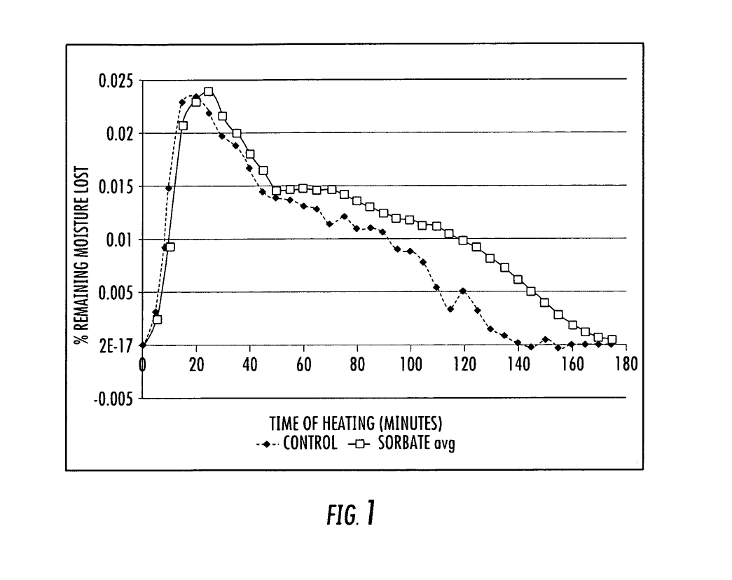

The control and Example 2 were applied to handsheet batches made according to TAPPI (Technical Association of the Pulp and Paper Industry) Test Method T-205 comprising 80% northern bleached hardwood and 20% northern bleached softwood kraft pulp. Test samples were applied at the rate of either 10 pounds solution per ton of pulp or 6 pounds of solution per ton of pulp. The handsheet drying rate was determined using a moisture analyzer. After couching each handsheet from the mold, a 100 cm² circle was cut using a precision die cutter. Each handsheet sample was placed into the moisture analyzer and dried at 150° C. The moisture analyzer recorded the weight every five seconds on a printout. Ten handsheets were prepared for each test sample and addition level and control experiment. The weight of each handsheet was recorded during the drying process and plotted against the time of heating. The slope, which shows the rate of weight loss representing the rate of moisture loss, of each test sample was determined. The slope was determined to normalize for moisture differences at the start for the different samples. The graph in FIG. 1 shows the average of all of the test sample slopes as well as the slope of the control (no stabilized potassium sorbate) tests.

The graph shows that after an initial induction period the rate of moisture loss in the treated samples is significantly higher than the rate of that of the control (not treated) sample. Since the input of energy is identical between test and control samples, this result demonstrates that after an initial induction period the test samples required significantly less energy to remove water than the control.

Titration of the resulting handsheets showed that the sorbate retention was greater than 75% for the handsheets made with the 42% sorbate treatment. The other samples did not have sufficient sorbate added to allow for accurate measurement of the sorbate remaining.

### Example 3

Aqueous solutions of potassium sorbate were prepared at 42%, 5% and 3% concentration and stabilized with manganese sulfate. Seven pounds of wet corn gluten (moisture content 67.6%) were weighed onto each of 4 baking sheets. Onto each sample of corn gluten, 9.5 grams of the sorbate solution or control (water) was sprayed using a pump sprayer. Each 9.5 gram treatment is equivalent to 6 pounds per ton or 0.3% treatment of the solution. The treatment of sorbate or water was, relative to the corn gluten, 0.13% (42% sorbate), 0.015% (5% sorbate), 0.009% (3% sorbate) and 0% (control).

Each treatment of corn gluten was placed into a polyethylene bag and heat sealed. The bags were placed in a tumble mixer and mixed for up to two hours to ensure even mixing. After mixing the bags were placed in a refrigerated cooler and left at 40° F. overnight. The next morning, the bags were placed in a circulating hot air oven and the temperature was raised to an even 72° F. throughout. The corn gluten was then placed into sealed plastic container for analysis.

Analysis was conducted in an impingement dryer set at 200° F. Seventy-five gram samples of each treatment were weighed and placed in circular metal pans. The bottom of the pans consisted of 70 mm mesh screen which allowed for air circulation while retaining the particles.

Samples were removed every three minutes for the first eighteen minutes and then every five minutes, weighed, and immediately placed back in the oven. Samples were heated until the equilibrium weight of approximately 28 grams. It is noted that the sorbate treated samples dried more quickly than the control and thus were brought to a lower moisture level during the heating test.

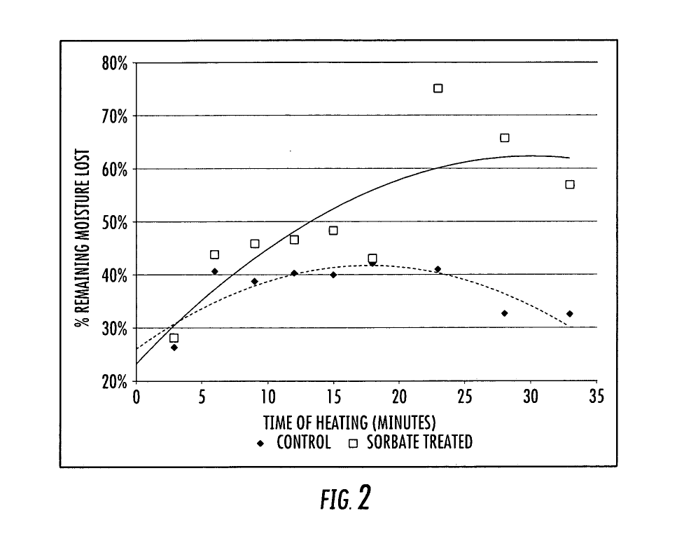

FIG. 2 shows the percent of the remaining moisture lost at each time period during the test. The data for the sorbate-treated samples were averaged as there were no significant differences found between the treatment levels.

During the early stage of drying, the rates at which water is removed is similar between treated and untreated samples. However, as the water content drops, the treated samples show similar or improved rates of de-watering, while the untreated sample shows that the rate of water removal declines.

### Example 4

Aqueous solutions were prepared containing varying combinations of potassium sorbate and/or manganese sulfate as shown in Table 2.

TABLE 2

| Sample # | % Potassium sorbate | % Manganese sulfate |
|---|---|---|
| 1 | 3 | 0.06 |
| 2 | 3 | 0 |
| 3 | 0 | 0.06 |
| 4 (control) | 0 | 0 |

Samples of wet corn gluten were treated and analyzed as described in Example 3. The treatment level of sorbate is at 0.009% relative to the wet corn gluten, while the treatment level of manganese sulfate is at 0.00018% relative to the wet corn gluten.

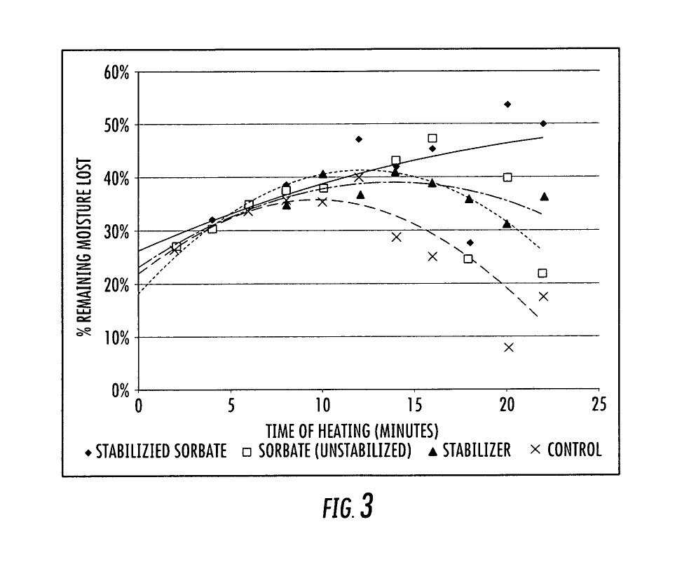

FIG. 3 shows the percent of the remaining moisture removed at each time period for the four treatments. During the early stage of drying, the rates at which water is removed is similar among all samples. However, as the water content drops, the sample treated with stabilized sorbate shows similar or improved rates of de-watering compared with the initial rate of water removal, while the other samples show a declining rate of water removal.

### Example 5

Aqueous solutions of potassium sorbate and manganese sulfate were prepared. The concentration of potassium sorbate varied from 1.3125-21%. The concentration of manganese sulfate varied from 0.00263-0.263%. Samples of wet corn gluten were treated and analyzed as described in Example 3. The treatment level at sorbate varied from 0.004-0.063% relative to the wet corn gluten, while the treatment level of manganese sulfate varied from 0.000008-0.0002% relative to the wet corn gluten.

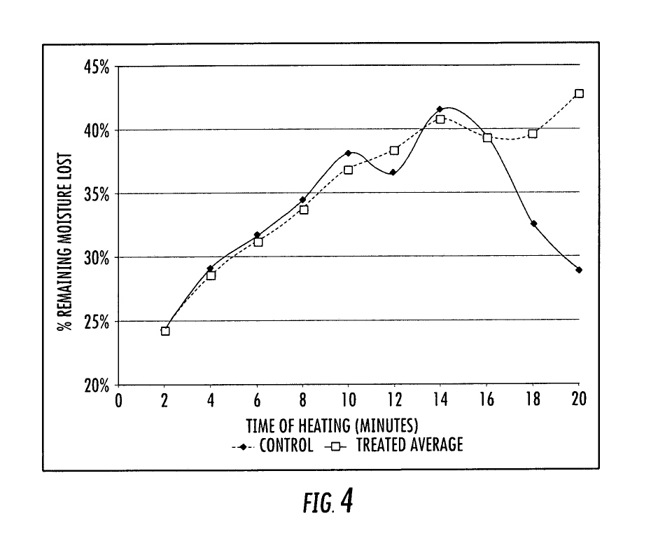

FIG. 4 shows the percent of the remaining moisture removed at each time period for the treated versus control samples. During the early stages of drying, the rates at which water is removed is similar among all samples. However, as the water content drops, the samples treated with stabilized sorbate shows similar or improved rates of de-watering compared with the initial rate of water removal, while the control show a declining rate of water removal.

### Example 6

Duplicate preparations of aqueous solutions of potassium sorbate were prepared at 6%, 3% and 1.5% concentration. One set of the preparations was stabilized with manganese sulfate at 0.0075% to 0.12%. The second set of preparations was not stabilized as a potassium sorbate only control. One set of aqueous solutions was prepared from 0.0075% to 0.12% manganese sulfate as a stabilizer only control. Seven pounds of wet corn gluten (moisture content 67.6%) were weighed onto each of 17 baking sheets. Onto each sample of corn gluten, 9.5 grams of the stabilized sorbate solution, the potassium sorbate only solution (control), the stabilizer only solution (control), or untreated control (water) was sprayed using a pump sprayer. Each 9.5 gram treatment is equivalent to 6 pounds per ton or 0.3% treatment of the solution. The treatment of sorbate or water varied from, relative to the corn gluten, 0.018% (6% sorbate) to 0% (control). The treatment of stabilizer varied from 0.000023% to 0.00036%. In all stabilized samples the ratio of sorbate to stabilizer was 50:1 (weight:weight).

Each treatment of corn gluten was placed into a polyethylene bag and heat sealed. The bags were placed in a tumble mixer and mixed for up to two hours to ensure even mixing. After mixing the bags were placed in a refrigerated cooler and left at 40° F. overnight. The next morning, the bags were placed in a circulating hot air oven and the temperature was raised to an even 72° F. throughout. The corn gluten was then placed into sealed plastic container for analysis.

Analysis was conducted in an impingement dryer set at 200° F. Seventy-five gram samples of each treatment were weighed and placed in circular metal pans. The bottom of the pans consisted of 70 mm mesh screen which allowed for air circulation while retaining the particles. Samples were removed every two minutes for twenty four minutes, weighed, and immediately placed back in the oven. Samples were heated until the equilibrium weight of approximately 30 grams was achieved. It is noted that the sorbate treated samples dried more quickly than the control and thus were brought to a lower moisture level during the heating test. The final moisture content was significantly (p=0.01) lower for the stabilized potassium sorbate than the water control, as shown in Table 3. The final moisture content was lower (p=0.05) for the stabilizer only, while the untreated potassium sorbate samples were not different from control (p<0.10).

TABLE 3

| | Final Corn Gluten | Difference from Untreated |
|---|---|---|
| Sample | Weight (g) | Control (p) |
| Stabilized potassium | 30.5 | p = 0.01 |
| sorbate | | |
| Potassium sorbate | 30.8 | p > 0.10 |
| Manganese sulfate | 30.7 | p = 0.05 |
| Water control | 31.0 | NA |

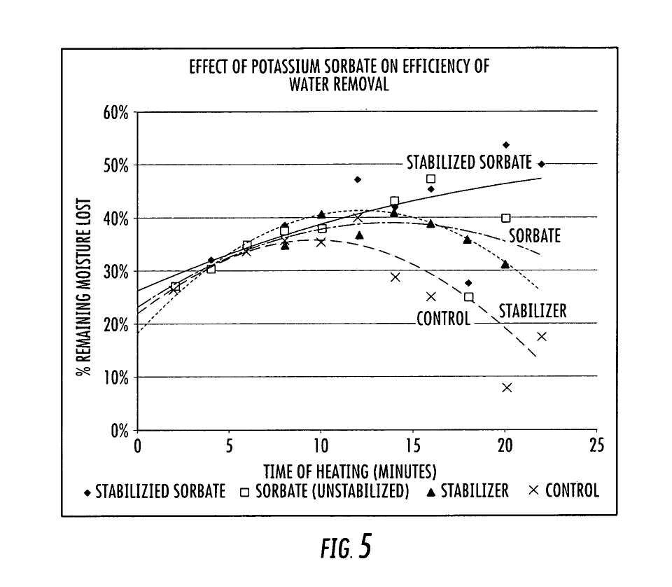

FIG. 5 shows the percent of the remaining moisture lost at each time period during the test. The data for the like-treated samples (stabilized potassium sorbate, potassium sorbate only, stabilizer only, and water control) were averaged as there were no significant differences found between the treatment levels. While the final moisture content for the potassium sorbate only treated samples (no stabilizer) was not significantly lower than that of control (no treatment), the amount of remaining moisture removed per unit time only diverges from that of the stabilized sorbate treatment after approximately 15 minutes of heating. This result is not unexpected as the function of the stabilizer is to ensure the efficacy of the potassium sorbate even after prolonged heating or other operations. Once the unexpected result of improved drying was observed following treatment with stabilized sorbate, the effect can be observed in the early stages of drying with sorbate that has not been stabilized. Thus, the improved drying rate effect is a result of the treatment with potassium sorbate, and the stabilization of the sorbate makes the improvement effective over a broad range of moisture contents, drying processes and substrates.

During the early stage of drying, the rates at which water is removed is similar between treated and untreated samples. However, as the water content drops, the stabilized potassium sorbate treated samples show similar or improved rates of de-watering, while the untreated water control shows that the rate of water removal declines. Unstabilized potassium sorbate shows much less effect on drying rate in this test. The high temperatures of the drying oven are likely to cause decomposition of the sorbate under these conditions, resulting in no significant improvement in drying rate as the moisture content drops and the cumulative amount of heat increases. The lesser improvement in drying rate observed by the addition of the manganese sulfate stabilizer is likely due to a change in the ionic strength of the system, which is known to affect surface charge and hence attraction of water to the matrix.

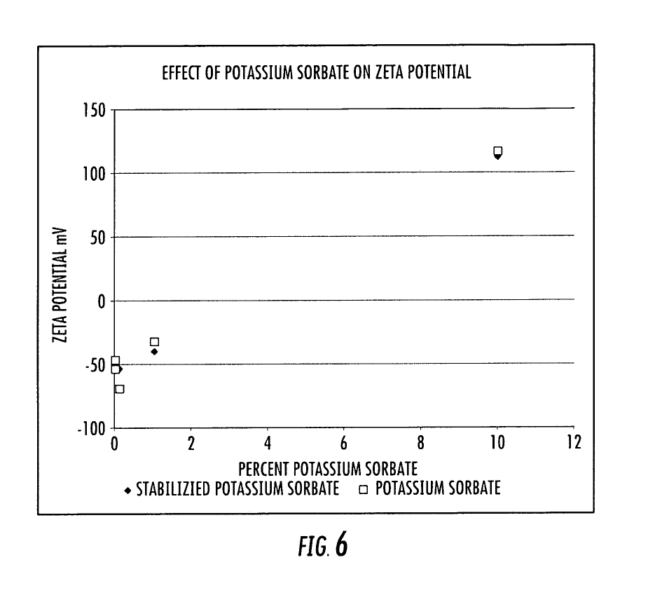

The possibility that the surface charge is altered by the addition of potassium sorbate was studied by observing the change in zeta potential of aqueous solutions containing from 0.0001% to 10% potassium sorbate. Concentrations below approximately 0.01% have no effect on the zeta potential and experimentation has shown those low concentrations to be ineffective for improving drying rates. FIG. 6 shows the change in zeta potential as the concentration of potassium sorbate is increased (samples are shown with no added sodium chloride; tests run using additional 0.01% sodium chloride were not different from un-supplemented samples and are not shown for clarity). As potassium sorbate concentration is increased, the zeta potential becomes less negative, suggesting that the addition of sorbate allows for the particles of the matrix to be more attracted to each other, and less attracted to the solvent (water), resulting in less energy required to remove water. In this experiment, the treatment was mild and hence the stabilizer is unnecessary to prevent oxidation of the sorbate and the non-stabilized as well as stabilized sorbate samples performed equally well.

### Example 7

Multiple preparations of aqueous solutions of potassium sorbate were prepared at 21%, 10.5%, 5.25%, 2,625% and 1.313% (weight:weight) concentration. The preparations were stabilized with manganese sulfate at 0.0026%, 0.0053%, 0.0105%, 0.021%, 0.028%, 0.0525%, 0.1204% and 0.2626% (weight:weight). The ratio of potassium sorbate to manganese sulfate in each of the preparations varied from 5:1 to 1000:1 (weight:weight). Seven pounds of wet corn gluten (moisture content 61.25%) were weighed onto each of 17 baking sheets. Onto each sample of corn gluten, 9.5 grams of the stabilized sorbate solution or untreated control (water) was sprayed using a pump sprayer. Each 9.5 gram treatment is equivalent to 6 pounds per ton or 0.3% treatment of the solution. The treatment of sorbate or water varied from, relative to the corn gluten, 0.063% (21% sorbate) to 0% (control). The treatment of stabilizer varied from 0.00078% to 0.079%.

Each treatment of corn gluten was placed into a polyethylene bag and heat sealed. The bags were placed in a tumble mixer and mixed for up to two hours to ensure even mixing. After mixing the bags were placed in a refrigerated cooler and left at 40° F. overnight. The next morning, the bags were placed in a circulating hot air oven and the temperature was raised to an even 72° F. throughout. The corn gluten was then placed into sealed plastic container for analysis.

Analysis was conducted in an impingement dryer set at 200° F. Seventy-five gram samples of each treatment were weighed and placed in circular metal pans. The bottom of the pans consisted of 70 mm mesh screen which allowed for air circulation while retaining the particles.

Samples were removed every two minutes for twenty four minutes, weighed, and immediately placed back in the oven. Samples were heated until the equilibrium weight of approximately 30 grams was achieved. It is noted that the sorbate treated samples dried more quickly than the control and thus were brought to a lower moisture level during the heating test. The final moisture content was significantly (p=0.01) lower for the stabilized potassium sorbate than the water control, as shown in Table 4. There were no significant differences between any of the treated samples so the results were averaged.

TABLE 4

| | Final Corn Gluten | Difference from Untreated |
|---|---|---|
| Sample | Weight (g) | Control (p) |
| Stabilized potassium | 29.3 | p < 0.01 |
| sorbate | | |
| Water control | 30.0 | NA |

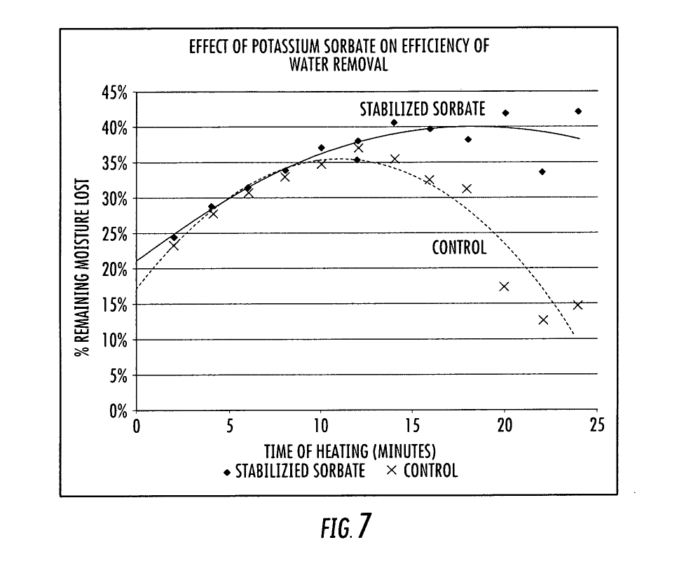

FIG. 7 shows the percent of the remaining moisture lost at each time period during the test.

During the early stage of drying, the rates at which water is removed is similar between treated and untreated samples. However, as the water content drops, the stabilized potassium sorbate treated samples show similar or improved rates of de-watering compared with the initial rate of water removal, while the untreated water control shows that the rate of water removal declines.

### Example 8

A single preparation of a pulp furnish of 80% northern hardwood and 20% northern softwood was prepared. The furnish was processed using a pilot paper machine at University of Wisconsin at Stevens Point, College of Natural Resources, Department of Paper Science and Engineering, Stevens Point, Wis., USA, using standard paper production techniques known to those skilled in the art. The pulp was manufactured into a paper 16.6875 inches wide, with a basis weight of approximately 100 grams/meter². The machine speed as well as all operating parameters were controlled to provide steady-state operating conditions. Measurements were conducted to determine the effects of treatment on the physical properties of the paper as well as the operation of the equipment.

The pulp slurry was treated with stabilized potassium sorbate at the stuffbox, and mixed well before processing. Treatment with an aqueous solution of 42% stabilized potassium sorbate was at the level of 2, 3, and 4 pounds per ton (wet weight sorbate solution per ton (dry weight) of pulp). This correlates to a treatment level of 0.042%, 0.063% and 0.084% (dry weight:dry weight) of potassium sorbate per ton of pulp. Chemical treatment was withdrawn between treatment levels and the equipment was operated until untreated (no chemical addition) equilibrium was re-established. These non-treatment samples were used as control (n=3) for evaluation purposes.

No adverse operational effects (such as foaming, precipitation, fouling, etc.) were observed during the operation of the equipment either with treatment or without. Testing of paper physical properties showed no adverse effects of chemical treatment related to Consistency, Tensile Strength, Stretch, Sheffield Smoothness, Tear Strength, Internal Bond Strength, Burst Strength and Formation.

Effects on drying efficiency were observed at three points during operation. During paper formation, the slurry is first spread onto a moving web and water is removed by simple gravity drainage. The water generated by gravity drains into the couch pit. Following gravity drainage, a vacuum is applied across the lightly formed web to pull more water out. The web is then rolled between two canisters to press addition water out, and finally is moved across a series of steam-heated canisters to drive the last remaining water off the now-formed paper. In the system here, the efficiency of water removal was measured at the couch pit, the vacuum flatbox, and by the amount of steam required to ensure complete paper drying. All samples were dried to the same finished moisture content (approximately 5% off the last canister of the steam dryer).

Table 5 summarizes the results of the test. The couch pit flow rate is reported as flow rate of the water during initial drainage. A higher value indicates faster drainage of water during the initial formation. The flatbox vacuum results have been averaged across six separate vacuum sections, while the dryer canister results are reported as the flow rate of condensate steam from the dryer canister. In the flatbox vacuum as well as dryer canister measurements, reduced levels are obtained when less energy is required for water removal. For all measurements, the percent improvement from control is reported as “%”. Note that the units are not similar across the various points of measurement and the results are therefore not additive.

TABLE 5

| Treatment | | | Flatbox | | Con- | |
|---|---|---|---|---|---|---|
| Level | Couch Pit | | Vacuum | | densate | |
| (pounds/ | Flow Rate | | (inches | | Flow | |
| ton) | (gal/min) | % | water) | % | (mL/min) | % |
| 0 | 27.5 | NA | 24.5 | NA | 3469 | NA |
| (Control) | | | | | | |
| 2 | 40.1 | +45.8 | 23.4 | −4.5% | 3423 | −2.1% |
| 3 | 35.7 | +29.8 | 21.5 | −12.2% | 3432 | −1.8% |
| 4 | 34.6 | +26.2 | 23.3 | −4.9% | 3319 | −5.1% |

All treatment levels resulted in significant reduction in energy usage. Removal of the water during early stages of drying was improved, but significant effects were seen in later stages also.

Having thus described certain embodiments of the present invention, it is to be understood that the invention defined by the appended claims is not to be limited by particular details set forth in the above description as many apparent variations thereof are possible without departing from the spirit or scope thereof as hereinafter claimed.

What is claimed is:

1. A method of controlling the amount of water in a polymer composition or substrate comprising the step of adding to the polymer composition or substrate a low molecular weight unsaturated fatty acid and optionally a stabilizer composition for preventing oxidation of the low molecular weight unsaturated fatty acid.

2. The method of claim 1 where the low molecular weight unsaturated fatty acid and/or its salt is sorbic acid and/or its salts.

3. The method of claim 1 wherein the substrate is selected from a group consisting of wood products and pulp, animal feed, distiller's grain, gypsum board, textiles, pharmaceuticals, concrete, cereals, pet food, snack foods, produce, dairy products, unprocessed and processed agricultural human foods, packaged human foods, tobacco, rubber, specialty polymers, minerals, coal, charcoal, and bakery products.

4. The method of claim 1, wherein the method of application is a pump spray application.

5. The method of claim 1, further comprising a surfactant wherein a level of said surfactant is between 0.5 and 10%.

6. The method of claim 5, wherein said surfactant is propylene glycol.

7. The method of claim 1 where said stabilizer composition comprises manganous ion.

8. The method of claim 7 wherein such manganous ion is in the weight ratio of between 8000:1 to 5:1 sorbate ion to manganous ion.

9. The method of claim 7 wherein said stabilizer is manganous sulfate monohydrate.

10. A method of reducing the amount of water in a polymer composition comprising the steps of:
a) adding to the polymer composition a low molecular weight unsaturated fatty acid and optionally a stabilizer composition for preventing oxidation of the low molecular weight unsaturated fatty acid, and
b) subjecting the polymer composition to conditions sufficient to remove water from the polymer composition.

11. The method of claim 10 where the low molecular weight unsaturated fatty acid and/or its salt is sorbic acid and/or its salts.

12. The method of claim 10, further comprising a surfactant wherein a level of said surfactant is between 0.5 and 10%.

13. The method of claim 12, wherein said surfactant is propylene glycol.

14. The method of claim 10 where said stabilizer composition comprises manganous ion.

15. The method of claim 14 wherein said manganous ion is in the weight ratio of between 8000:1 to 5:1 sorbate ion to manganous ion.

16. The method of claim 14 wherein said stabilizer is manganous sulfate monohydrate.

17. A composition for applying to a substrate or adding to a polymer composition to facilitate water removal or to prevent water adsorption, the composition comprising a low molecular weight unsaturated fatty acid and a stabilizer composition for preventing oxidation of the low molecular weight fatty acid.

18. The composition of claim 17 where the low molecular weight unsaturated fatty acid and/or its salt is sorbic acid and/or its salts.

19. The composition of claim 17, further comprising a surfactant wherein a level of said surfactant is between 0.5 and 10%.

20. The composition of claim 19, wherein surfactant is at a level of about 1.5%.

21. The composition of claim 19, wherein said surfactant is propylene glycol.

22. The composition of claim 17 where said stabilizer composition comprises manganous ion.

23. The composition of claim 22 wherein such manganous ion is in the weight ratio of between 8000:1 to 5:1 sorbate ion to manganous ion.

24. The composition of claim 22 wherein said stabilizer is manganous sulfate monohydrate.
